(19) United States
(12) Patent Application Publication
(10) Pub. No.: US 2009/0124838 A1
(43) Pub. Date: May 14, 2009

(54) LIQUID CRYSTALLINE STYRYL DERIVATIVE, PROCESS OF PREPARING SAME, AND LIQUID CRYSTAL SEMICONDUCTOR DEVICE USING SAME

(75) Inventor: Yuichiro Haramoto, Yamanashi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignees: YAMANASHI UNIVERSITY, Kofu-shi, Yamanashi (JP); NIPPON CHEMICAL INDUSTRIAL CO., LTD., Tokyo (JP)

(21) Appl. No.: 12/279,406

(22) PCT Filed: Jan. 26, 2007
(86) PCT No.: PCT/JP2007/051251

§ 371 Date: Aug. 14, 2008

(30) Foreign Application Priority Data
Feb. 14, 2006 (JP) 2006-037149

(51) Int. Cl. C07C 15/52

(52) U.S. Cl. 585/25; 585/435

(57) ABSTRACT

A liquid crystalline styryl derivative represented by general formula (1):

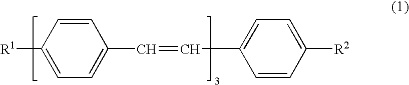

wherein R¹ and R², which may be the same or different,
each represent a straight-chain or branched alkyl group,
a straight-chain or branched alkoxy group, a cyano group,
a nitro group, F, —C(O)O(CH₂)ₘ—CH₃, —C(O)—(CH₂)ₘ—CH₃,
or general formula (2);

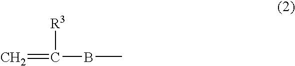

wherein R³ represents a hydrogen atom or a methyl group;
B represents —(CH₂)ₘ—, —(CH₂)ₘ—O—, —CO—O—(CH₂)ₘ—,
—CO—O—(CH₂)ₘ—O—, —C₆H₄—CH₂—O— or —CO—; and m represents
an integer of 1 to 18.
   R¹ and R², which may be the same or different, each preferably represent a branched alkyl or alkoxy group represented by CH₃—(CH₂)ₓ—CH(CH₃)—(CH₂)_{y}—CH₂— or CH₃—(CH₂)ₓ—CH(CH₃)—(CH₂)_{y}—CH₂—O—, respectively, wherein x is an integer of 0 to 7, and y is an integer of 0 to 7. The styryl derivative is suitable for use as an organic semiconductor material.

### TECHNICAL FIELD

This invention relates to a liquid crystalline styryl derivative useful as an organic electroluminescence (hereinafter “EL”) material or an organic semiconductor material, e.g., of a thin film transistor (hereinafter “TFT”) or a memory device. It also relates to a process of preparing the styryl derivative and a liquid crystal semiconductor device using the styryl derivative.

### BACKGROUND ART

Organic semiconductors have been attracting attention as semiconductor materials taking the place of silicon and compound semiconductors. Production of semiconductor devices using conventional semiconductors necessarily involves high vacuum and high temperature processing operations, which has made cost reduction difficult. In contrast, use of organic semiconductor materials will make it feasible to produce semiconductor devices through a simple processing operation such as coating with a semiconductor solution or vacuum evaporation under room temperature conditions.

The inventors of the present invention previously found that a liquid crystalline styryl derivative represented by general formula shown below and having a smectic phase as a liquid crystal phase develops excellent charge transport properties without requiring photoexcitation with a voltage applied while it is in a smectic phase or in a solid phase as a result of phase change from the smectic phase and proposed applying the styryl derivative as an organic EL material or to organic semiconductor devices, such and TFTs (see, e.g., Patent Document 1 to 5).

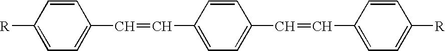

where R is an organic group such as an alkyl group or an alkoxy group

Compared with inorganic materials, organic materials, being a molecular substance, are generally more sensitive to light, heat, the air (O₂ and H₂O), and the like and more liable to undergo chemical reaction to decompose. This poses a serious problem in using an organic substance as a material. Material decomposition by light, oxygen, etc., however slight it may be, can greatly affect the performance, especially electrical characteristics. It has therefore been demanded to develop an endurable compound that withstands use in sites near electrodes which undergo strong electric stimuli.

Apart from the above described styryl derivative, the inventors proposed using a styryl derivative having four repeating styryl groups as a luminescent substance for organic EL devices (see Patent Document 6). Although the styryl derivative is characterized by luminescence at longer wavelengths than blue, it has insufficient solubility in some solvents.

### Patent Document 1: JP 2004-6271A

### Patent Document 2: US 2006/255318A1

### Patent Document 3: US 2006/278848A1

### Patent Document 4: JP 2004-311182A

### Patent Document 5: JP 2005-142233A

### Patent Document 6: JP 2005-272351A

### DISCLOSURE OF THE INVENTION

### Problem to be Solved by the Invention

An object of the present invention is to provide a novel liquid crystal compound suited for use even in a site of organic semiconductor devices where durability is required.

### Means for Solving the Problem

The invention achieves the object by the provision of a liquid crystalline styryl derivative represented by general formula (1):

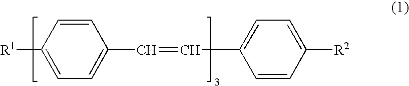

wherein R¹ and R², which may be the same or different,
each represent a straight-chain or branched alkyl group,
a straight-chain or branched alkoxy group, a cyano group,
a nitro group, F, —C(O)O(CH₂)ₘ—CH₃, —C(O)—(CH₂)ₘ—CH₃,
or general formula (2) below;

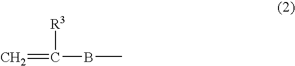

wherein R³ represents a hydrogen atom or a methyl group;
B represents —(CH₂)ₘ—, —(CH₂)ₘ—O—, —CO—O—(CH₂)ₘ—, —CO—O—(CH₂)ₘ—O—, —C₆H₄—CH₂—O— or —CO—; and m represents
an integer of 1 to 18.

The invention also provides a preferred process of preparing the styryl derivative. The process comprises causing a 4-styrylbenzaldehyde compound represented by general formula (3) to react with a phosphonium salt represented by general formula (4):

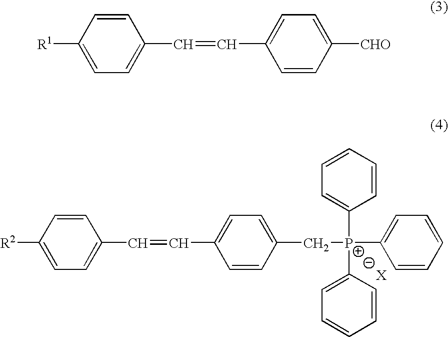

wherein R¹ and R² are as defined above; and X represents a halogen atom.

The invention also provides a liquid crystal semiconductor device having a liquid crystal material comprising the liquid crystalline styryl derivative.

### BRIEF DESCRIPTION OF THE DRAWINGS

FIG. 1 schematically illustrates a cross-sectional structure of an organic EL device as one embodiment of the liquid crystal semiconductor device of the invention.

FIG. 2 schematically illustrates a cross-sectional structure of an organic EL device as another embodiment of the liquid crystal semiconductor device of the invention.

FIG. 3 schematically illustrates a cross-sectional structure of a TFT device as still another embodiment of the liquid crystal semiconductor device of the invention.

FIG. 4 schematically illustrates a cross-sectional structure of a TFT device as yet another embodiment of the liquid crystal semiconductor device of the invention.

FIG. 5 is a graph showing the voltage vs. current relationship of a device using an electroconductive liquid crystal material containing the styryl derivative prepared in Example 1.

### BEST MODE FOR CARRYING OUT THE INVENTION

The liquid crystalline styryl derivative according to the invention is a liquid crystal compound having a long, linear conjugate structure. The styryl derivative of the invention exhibits a smectic phase in its liquid crystal state. The styryl derivative of the invention is characterized by having three repeating styryl groups in general formula (1). Owing to this characteristic, the liquid crystal styryl derivative has excellent durability. Compared with a compound having the same skeleton as the general formula (1) but having two repeating styryl groups, the styryl derivative of the invention is superior in electrical stability represented by, for example, small electrical activation energy. The styryl derivative of the invention is also superior to the compound with two repeating styryl groups in solubility in various solvents.

In general formula (1), R¹ and R², which may be the same or different, each represent a straight-chain or branched alkyl group, a straight-chain or branched alkoxy group, a cyano group, a nitro group, F, —C(O)O(CH₂)ₘ—CH₃, —C(O)—(CH₂)ₘ—CH₃, or a group having an unsaturated bond represented by general formula (2). The alkyl group preferably has 1 to 18 carbon atom. Examples of the alkyl group are methyl, ethyl, butyl, pentyl, hexyl, octyl, dodecyl, pentadecyl, and octadecyl. Preferred of them are those having 4 to 18 carbon atoms. In particular, the compound in which the alkyl group is a branched one represented by CH₃—(CH₂)ₓ—CH(CH₃)—(CH₂)_{y}—CH₂— (wherein x is an integer of 0 to 7, and y is an integer of 0 to 7), especially an isobutyl group (x=y=0), is preferable because of increased solubility in various solvents.

The alkoxy group, which is represented by general formula CₙH₂ₙ₊₁O—, is preferably one in which n is an integer of 1 to 20, more preferably 4 to 18. Examples of the alkoxy group include methoxy, ethoxy, butoxy, pentoxy, hexyloxy, octyloxy, dodecyloxy, pentadecyloxy, and octadecyloxy. In particular, the compound in which the alkoxy group is a branched one represented by CH₃—(CH₂)ₓ—CH(CH₃)—(CH₂)_{y}—CH₂—O— (wherein x is an integer of 0 to 7, and y is an integer of 0 to 7), especially an isobutoxy group (x=y=0) is preferable because of increased solubility in various solvents.

In formulae —C(O)O(CH₂)ₘ—CH₃ and —C(O)—(CH₂)ₘ—CH₃, m is preferably an integer of 1 to 18, more preferably an integer of 6 to 14.

In the group having an unsaturated bond as represented by general formula (2), R³ represents a hydrogen atom or a methyl group, and B represents —(CH₂)ₘ—, —(CH₂)ₘ—O—, —CO—O—(CH₂)ₘ—, —CO—O—(CH₂)ₘ—O—, —C₆H₄—O— or —CO—. The symbol m preferably represents an integer of 1 to 18, more preferably an integer of 6 to 14.

In general formula (1), R¹ and R² may represent the same group or different groups. It is preferred that at least one of R¹ and R² is a straight-chain or branched alkyl or alkoxy group, particularly an isobutyl or an isobutoxy group. It is also preferred that at least one of R¹ and R² is a straight-chain heptyl group or a straight-chain decyloxy group.

The liquid crystal styryl derivative of general formula (1) may be a cis-isomer, a trans-isomer or a mixture thereof.

The liquid crystalline styryl derivative of general formula (1) is conveniently prepared by causing a 4-styrylbenzaldehyde compound represented by general formula (3) and a phosphonium salt represented by general formula (4) to react with each other.

Processes for preparing the starting compounds used in the invention, i.e., the 4-styrylbenzaldehyde compound of general formula (3) and the phosphonium salt of general formula (4) are described, e.g., in WO2004/85398A1 and US2006/255318A1.

In the preparation of the styryl derivative of general formula (1) in which R¹ and R² are each an isobutyl group, for instance, 4-(4-isobutylstyryl)benzaldehyde and 4-(4-isobutylstyryl)benzylphosphonium bromide are used as compounds of general formulae (3) and (4), respectively.

More specifically, the liquid crystalline styryl derivative of general formula (1) in which both R¹ and R² are an isobutyl group can be obtained by, for example, synthesizing compounds (8) to (17) in accordance with reaction scheme 1 below which starts with 4-isobutylbenzaldehyde.

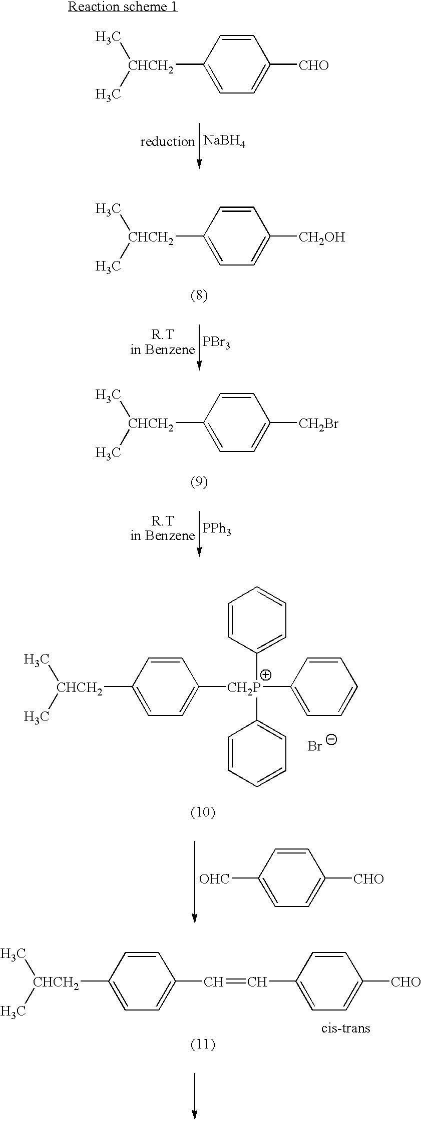

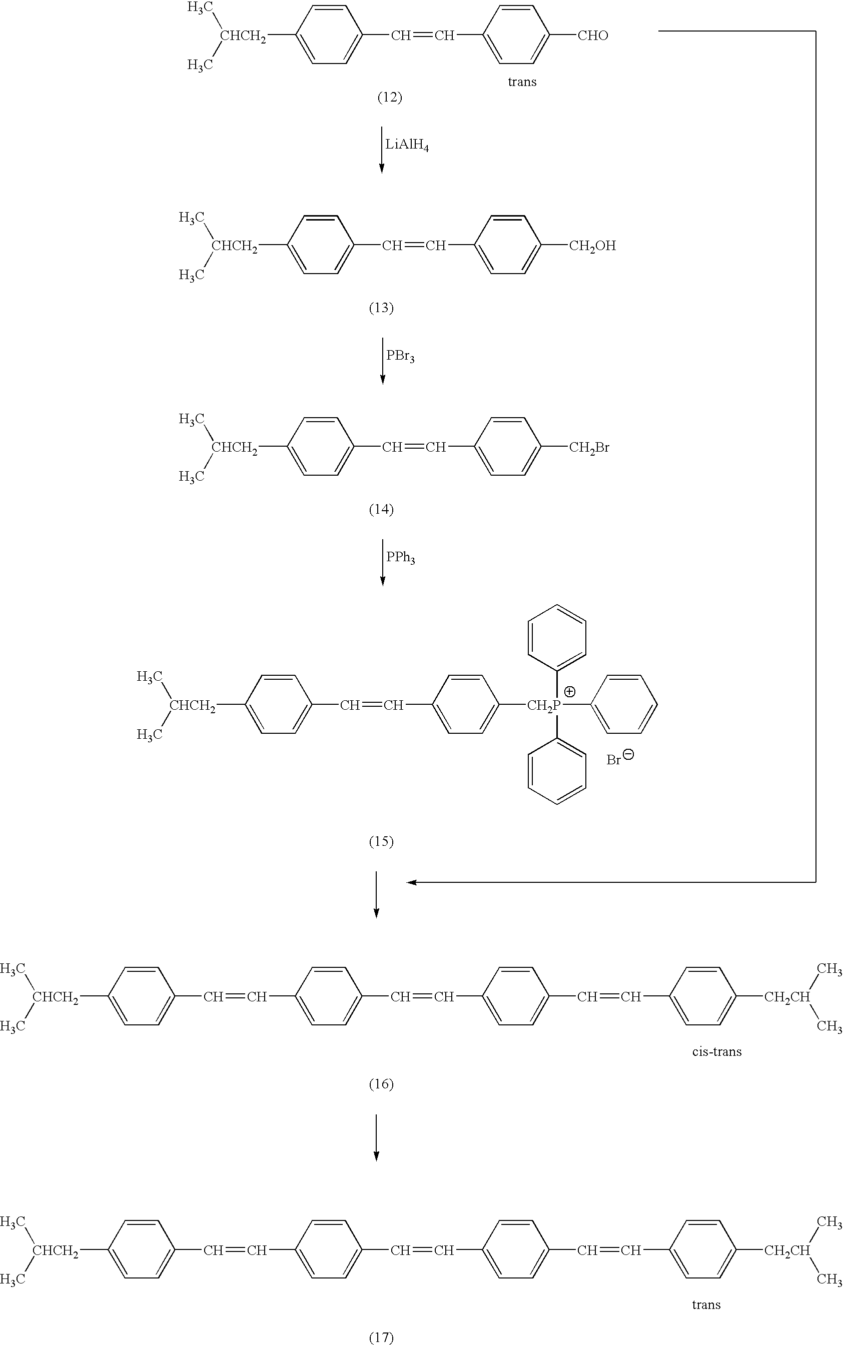

In reaction scheme 1, a base (e.g., NaBH₄) is allowed to act on 4-isobutylbenzaldehyde in a methanol solvent to obtain 4-isobutylbenzyl alcohol (8). The 4-isobutylbenzyl alcohol (8) is caused to react with phosphorus tribromide in benzene at room temperature to obtain 4-isobutylbenzyl bromide (9), which is then caused to react with triphenylphosphine in benzene at room temperature to obtain 4-isobutylbenzylphosphonium bromide (10). Compound (10) and terephthalaldehyde are allowed to react in methanol at 50° C. to obtain 4-(4-isobutylstyryl)benzaldehyde (11).

The resulting compound (11) is a cis-trans mixture. If necessary, the cis-trans mixture can be converted to the corresponding trans-form (12) by causing iodine to act thereon in toluene or xylene under reflux. The iodine is preferably added in an amount of 0.001 to 0.1 mol, more preferably 0.005 to 0.01 mol, per mole of compound (11).

The heating temperature is preferably 100° C. to 180° C., more preferably 130° C. to 150° C. Compound (11) and/or compound (12) are included in the 4-styrylbenzaldehyde compound of general formula (3).

A base (e.g., LiAlH₄) is caused to act on the resulting trans compound (12) in a solvent such as an ether or an alcohol to obtain 4-(4-isobutylstyryl)benzyl alcohol (13). Compound (13) and phosphorus tribromide are caused to react in benzene at room temperature to obtain 4-(4-isobutylstyryl)benzyl bromide (14). Compound (14) is caused to react with triphenylphosphine in benzene at room temperature to obtain 4-(4-isobutylstyryl)benzylphosphonium bromide (15), which is included in the phosphonium salt of general formula (4).

(compound (11) or (compound (12)), preferably the trans-form (12) of 4-(4-isobutylstyryl)benzaldehyde, and the 4-(4-isobutylstyryl)benzylphosphonium bromide (15) are allowed to react in a solvent such as an alcohol in the presence of a base.

Examples of the base that can be used include metal hydrides such as sodium hydride; amines such as trimethylamine and triethylamine; alkali metal hydroxides such as potassium hydroxide and sodium hydroxide; alkoxides such as sodium methoxide, potassium methoxide, sodium ethoxide, and potassium ethoxide; pyridine, potassium cresolate, and alkyllithiums. These bases may be used either individually or a combination of two or more thereof. The amount of the base to be added is 0.8 to 5 mol, preferably about 1 mol, per mole of compound (15). Compound (15) is used in an amount of 0.9 to 1.1 mol, preferably about 1 mol, per mole of compound (12). The reaction is carried out at 0° C. to 150° C., preferably 30° C. to 80° C., for at least 5 hours, preferably 10 to 30 hours. After completion of the reaction, the reaction mixture is filtered, and the filter cake is optionally washed and dried to give a styrene derivative (16).

The styryl derivative (16) is a cis-trans mixture. If necessary, the cis-trans mixture can be converted to the corresponding trans-form (17) by causing iodine to act thereon in toluene or xylene under reflux. The iodine is preferably added in an amount of 0.001 to 0.1 mol, more preferably 0.005 to 0.01 mol, per mole of compound (15). The heating temperature is preferably 100° C. to 180° C., more preferably 130° C. to 150° C.

Various styryl derivatives obtained in that way are superior in electrical stability represented by, for example, small electrical activation energy as compared with compounds having the same skeleton as the general formula (1) but having two repeating styryl groups. In applications as a luminescent substance to organic EL devices, the styryl derivatives emit light of about 430 nm, whereas the compounds having two repeating styryl groups emit blue light having shorter wavelengths (about 420 nm) than the luminescence wavelengths of the styryl derivatives of the invention. The styryl derivatives of the invention are useful as a charge transport material in photosensors, photoconductors, spatial modulators, TFTs, and electrophotographic photoreceptors, in which applications the charge transport properties of the styryl derivatives are made use of. The styryl derivatives are also useful as a material used in photolithography, solar cells, nonlinear optics, organic semiconductor condensers, and other sensors and so on. The liquid crystalline styryl derivative of the invention is especially useful as an organic EL material and an organic semiconductor material for TFTs, memory devices, etc.

The liquid crystalline styryl derivative represented by general formula (1) develops electroconductivity with a voltage applied while it is in a liquid crystal phase of a smectic phase or in a solid phase resulting from phase transition from the smectic phase. The liquid crystalline styryl derivatives can be used either individually or as a combination of two or more thereof. The liquid crystalline styryl derivative may be used in combination with other liquid crystal compounds having a long linear conjugate structure, such as those represented by general formulae (6a) through (6f):

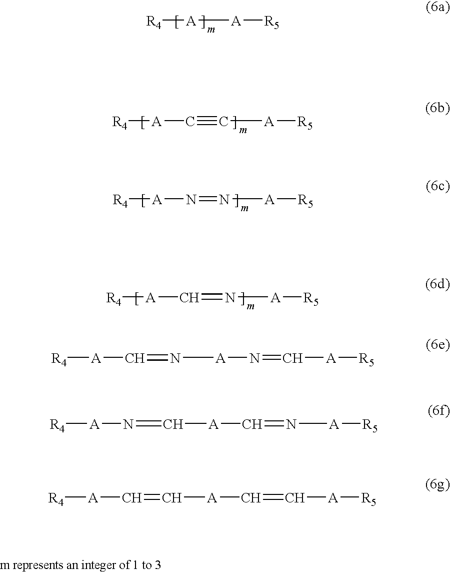

In general formulae (6a) to (6g) representing the liquid crystal compounds having a long linear conjugate structure, R₄ and R₅ each represent a straight-chain or branched alkyl group or a straight-chain or branched alkoxy group. The alkyl group preferably has 3 to 20 carbon atom. Examples of the alkyl group are butyl, hexyl, heptyl, octyl, nonyl, decyl, dodecyl, pentadecyl, and octadecyl. In particular, the compound in which the branched alkyl group is represented by CH₃—(CH₂)ₓ—CH(CH₃)—(CH₂)_{y}—CH₂— (wherein x is an integer of 0 to 7, and y is an integer of 0 to 7) has increased solubility in various solvents. The alkoxy group, which is represented by general formula CₙH₂ₙ₊₁O—, is preferably one in which n is 3 to 20. In particular, the compound in which the branched alkoxy group is represented by CH₃—(CH₂)ₓ—CH(CH₃)—(CH₂)_{y}—CH₂—O— (wherein x is an integer of 0 to 7, and y is an integer of 0 to 7) has increased solubility in various solvents. Symbol A in the formulae (6a) to (6g) is exemplified by the groups represented by formulae (7a) to (7e) as shown below.

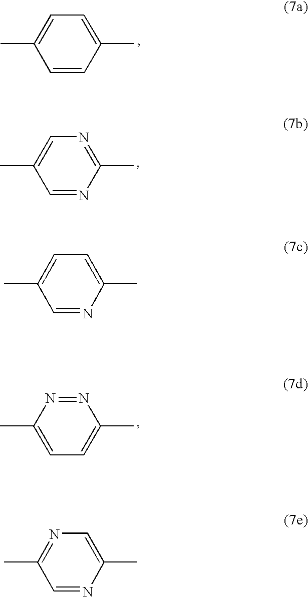

The liquid crystal semiconductor device according to the present invention is characterized by having a liquid crystal material containing the liquid crystalline styryl derivative represented by general formula (1). The liquid crystal material exhibits a smectic phase as a liquid crystal phase and contains at least one liquid crystal styryl derivative of general formula (1) in a proportion usually of at least 5% by weight, preferably 30% by weight or more, more preferably 70% by weight or more.

Other liquid crystal compounds that may be used in combination with the liquid crystal styryl derivative of general formula (1) are exemplified by those having a long linear conjugate structure represented by general formulae (6a) to (6g) described supra.

The liquid crystal material may be prepared by (a) dissolving at least one styryl derivative of general formula (1) and other necessary components in a solvent, followed by solvent removal by heating under reduced pressure or the like, (b) mixing at least one styryl derivative of general formula (1) and other necessary components and heating the mixture to melt, or (c) subjecting the mixture to sputtering, vacuum deposition, oblique vacuum deposition, and the like. Vacuum deposition and oblique vacuum deposition will hereinafter be referred to as (oblique) vacuum deposition. The liquid crystal material is preferably a thin film formed by (oblique) vacuum deposition to a thickness of 100 nm to 1000 μm. The preference is because such a thin film as deposited has a sparse structure so that the molecules are easy to re-align by heat treatment. Therefore, when a liquid crystal material formed by vacuum deposition is heat treated to once form a liquid crystal phase of a smectic phase as described later, it has improved memory of the liquid crystal molecular alignment of the smectic phase as compared with liquid crystal materials formed by other processes. Therefore, even after the temperature is returned to room temperature, the resulting solid-state liquid crystal material retains the molecular alignment of the smectic phase almost perfectly. This solid-state liquid crystal material serves as a liquid crystal material having excellent electroconductivity.

The above described liquid crystal material of thin film form is preferably subjected to heat treatment at a temperature for inducing a phase transition to a liquid crystal phase of a smectic phase in an inert gas atmosphere such as nitrogen, argon or helium to have a controlled molecular alignment and thereby to have excellent electroconductivity.

The heat treating temperature for inducing a phase transition to a smectic phase is selected from the range in which the liquid crystal material exhibits per se a liquid crystal phase of a smectic phase. The heat treating time is not particularly limited and ranges usually from about 1 to 60 minutes, preferably from about 1 to 10 minutes.

The liquid crystal semiconductor device of the invention is useful as an organic EL device or a TFT device.

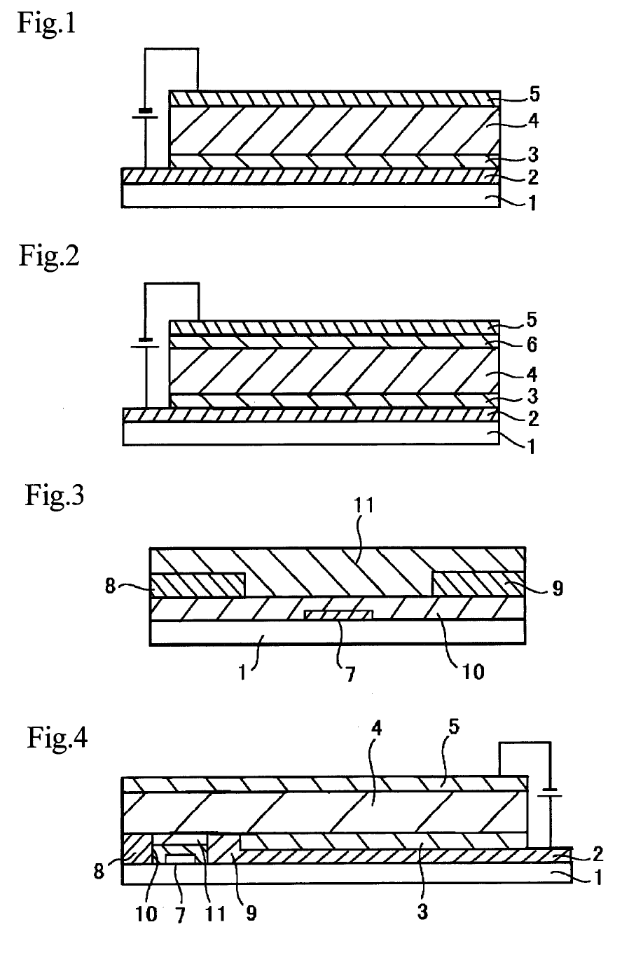

The liquid crystal semiconductor device of the invention will be described by way of the drawing. FIGS. 1 through 4 are each a schematic of an embodiment of the liquid crystal semiconductor device of the invention. The device of FIG. 1 is composed of a transparent substrate 1, a positive electrode 2, a buffer layer 3, an electroconductive liquid crystal layer 4, and a negative electrode 5, stacked one on top of another in the order described. This device is especially suited for use as an organic EL device. The substrate 1 is usually a glass plate or the like, which is commonly used in organic EL devices. The positive electrode 2 is made of a material having a large work function and optionally transparent for extracting light. An ITO film is suitable, for example. The negative electrode 5 is formed of a thin film of a metal having a small work function, such as Al, Ca, LiF, Mg, or an alloy thereof.

The electroconductive liquid crystal layer 4 is formed of the liquid crystal material of the invention. Since the styryl derivative of general formula (1) per se has green light emitting properties, the electroconductive liquid crystal layer 4 has a function as a luminescent layer or a carrier transport layer. Optionally, a small amount of a luminescent material may be incorporated into the liquid crystal layer as long as the solid state resulting from a phase transition from the smectic phase of the liquid crystal material may be retained. Examples of usable luminescent materials include diphenylethylene derivatives, triphenylamine derivatives, diaminocarbazole derivatives, benzothiazole derivatives, benzoxazole derivatives, aromatic diamine derivatives, quinacridone compounds, perylene compounds, oxadiazole derivatives, coumarin compounds, anthraquinone derivatives, dyes for laser oscillation (e.g., DCM-1), various metal complexes, low molecular fluorescent dyes, and high molecular fluorescent materials.

It is particularly preferred that the electroconductive liquid crystal layer 4 in the liquid crystal semiconductor device of the invention is one obtained by depositing components of the liquid crystal material either simultaneously or separately by (oblique) vacuum evaporation in a room temperature range (from 5° C. to 40° C.) and then heating the deposited layer to a temperature at which the liquid crystal material undergoes phase change to a smectic phase in an inert gas atmosphere such as nitrogen, argon or helium.

The buffer layer 3, which is optionally provided to reduce the energy barrier to hole injection from the positive electrode 2. Materials of the buffer layer 3 include copper phthalocyanine, poly(3,4-ethylenedioxythiophene-polystyrene sulfonate (hereinafter abbreviated as PEDOT-PSS), phenylamines, star-burst amines, vanadium oxide, molybdenum oxide, ruthenium oxide, aluminum oxide, amorphous carbon, polyaniline, and polythiophene derivatives. A buffer layer may be provided on the negative electrode 5 side to promote electron injection.

FIG. 2 is a schematic of another embodiment of the liquid crystal semiconductor device of the invention which is suited for use as an organic EL device. The device shown is composed of a transparent substrate 1, a positive electrode 2, a buffer layer 3, a liquid crystal compound layer 4, an organic luminescent layer 6, and a negative electrode 5, stacked one on top of another in the order described. This device is different from the one shown in FIG. 1 in that the luminescent layer 6 is not an electroconductive liquid crystal layer. The luminescent layer 6 is made of various conventional organic luminescent materials including diphenylethylene derivatives, triphenylamine derivatives, diaminocarbazole derivatives, benzothiazole derivatives, benzoxazole derivatives, aromatic diamine derivatives, quinacridone compounds, perylene compounds, oxadiazole derivatives, coumarin compounds, anthraquinone derivatives, dyes for laser oscillation (e.g., DCM-1), various metal complexes, low molecular fluorescent dyes, and high molecular fluorescent materials.

In this embodiment, the electroconductive liquid crystal layer 4 is formed of the liquid crystal material of the invention. It is particularly preferred that the electroconductive liquid crystal layer 4 is one obtained by depositing components of the liquid crystal material either simultaneously or separately by (oblique) vacuum evaporation in a room temperature range (from 5° C. to 40° C.) and heating the deposited layer to a temperature at which the liquid crystal material undergoes phase change to a smectic phase in an inert gas atmosphere such as nitrogen, argon or helium.

In the present embodiment, the electroconductive liquid crystal layer 4 functions primarily as a carrier transport layer. Compared with conventional amorphous organic compounds, the liquid crystal layer 4 has high carrier transport properties. This will allow for increasing the layer thickness and increasing the carrier injection efficiency thereby to reduce the driving voltage.

In the above illustrated organic EL devices, the electroconductive liquid crystal layer 4 may be designed to have a thickness of 100 nm to 100 μm.

FIG. 3 is a schematic of an embodiment of the liquid crystal semiconductor device of the invention which is suited for use as a TFT device. The TFT is a field effect transistor having a source 8 and a drain 9 formed on a substrate 1 in a facing relationship with each other with a gate 7 therebetween. The TFT has an insulator layer 10 covering the gate 7 and a channel 11 formed outside the insulator layer 10, through which current may flow between the source 8 and the drain 9. The substrate 1 is made of an insulating material such as an inorganic material (e.g., glass or sintered alumina), a polyimide film, a polyester film, a polyethylene film, a polyphenylene sulfide film, or a poly-p-xylene film. The gate 7 is formed of an organic material such as polyaniline or polythiophene; a metal such as gold, platinum, chromium, palladium, aluminum, indium, molybdenum or nickel, or an alloy thereof; polysilicon, amorphous silicon, tin oxide, indium oxide, indium, or tin oxide. The insulator layer 10 is preferably formed by applying an organic material. Examples of the organic material include polychloroprene, polyethylene terephthalate, polyoxymethylene, polyvinyl chloride, polyvinylidene fluoride, cyanoethylpullulan, polymethyl methacrylate, polysulfone, polycarbonate, and polyimide. The source 8 and the drain 9 are formed of gold, platinum, a transparent conductor layer (e.g., indium tin oxide or indium zinc oxide), or the like. The channel 11 is formed of the liquid crystal material of the invention. It is preferred that the channel 11 is one formed by depositing components of the liquid crystal material either simultaneously or separately by (oblique) vacuum evaporation in a room temperature range (from 5° C. to 40° C.) and heating the deposited layer to a temperature at which the liquid crystal material undergoes phase change to a smectic phase in an inert gas atmosphere such as nitrogen, argon or helium. Where necessary, the liquid crystal material may be used in combination with an electron accepting substance or an electron donating substance to emphasize the p-type or n-type properties. When an electric field induced by the gate 7 is applied to the channel 11 formed of the liquid crystal material, the amount of the holes or electrons inside the channel region 11 can be controlled, whereby the channel 11 adds the function as a switching device. When the insulator layer 10 made of, e.g., polyimide is rubbed and then the electroconductive liquid crystal layer is formed thereon, the resulting electroconductive liquid crystal layer exhibits improved molecular alignment, thereby to reduce the operating voltage or increase the operating speed of the TFT. The rubbing direction is preferably perpendicular to the current flow direction between the source 8 and the drain 9 (e.g., the line connecting their centers). In this case, the side chains of the liquid crystal compound molecules having a long linear conjugate structure are aligned perpendicular to the current flow between the source and the drain with the conjugate core portions of the molecules aligned closely. As a result, the channel 11 has remarkably improved carrier transport properties, exhibiting electroconductivity at the level of semiconductors such as silicon.

FIG. 4 is a schematic cross-sectional structure of an organic EL device having a TFT as an embodiment of the liquid crystal semiconductor device according to the invention.

The device of FIG. 4 has an EL device main body and a TFT as a switching element formed on a common substrate 1. The TFT used here is the above-described TFT. That is, a source 8 and a drain 9 are formed adjacent the EL device main body on the substrate 1 in a facing relationship with each other with a gate 7 therebetween. An insulator layer 10 is provided to cover the gate 7, and a channel 11 is formed outside the insulator layer 10, through which current may flow between the source 8 and the drain 9. The channel 11 is formed of the above described liquid crystal material. A matrix pixel driving scheme being employed, the gate 7 and the source 8 are connected to X and Y signal lines, respectively, and the drain 9 is connected to one of the electrodes of the EL device (positive electrode in this particular example).

The liquid crystal material making the channel 11 may be the same as used to make the electroconductive liquid crystal layer 4 of the EL device main body, and the channel 11 and the electroconductive liquid crystal layer 4 may be formed integrally. As a result, an organic EL device main body and a TFT can be formed at the same time in the production of an active matrix type organic EL device, so that the production cost will be reduced further.

It is preferred that the channel 11 and the electroconductive liquid crystal layer 4 is formed by depositing components of the liquid crystal material either simultaneously or separately by (oblique) vacuum evaporation in a room temperature range (from 5° C. to 40° C.) and heating the deposited layer to a temperature at which the liquid crystal material undergoes phase transition to a smectic phase in an inert gas atmosphere such as nitrogen, argon or helium.

### EXAMPLES

The present invention will now be illustrated in greater detail with reference to Examples, but it should be understood that the invention is not limited thereto.

### Example 1

Compound (16) and compound (17) were synthesized in accordance with the following reaction scheme:

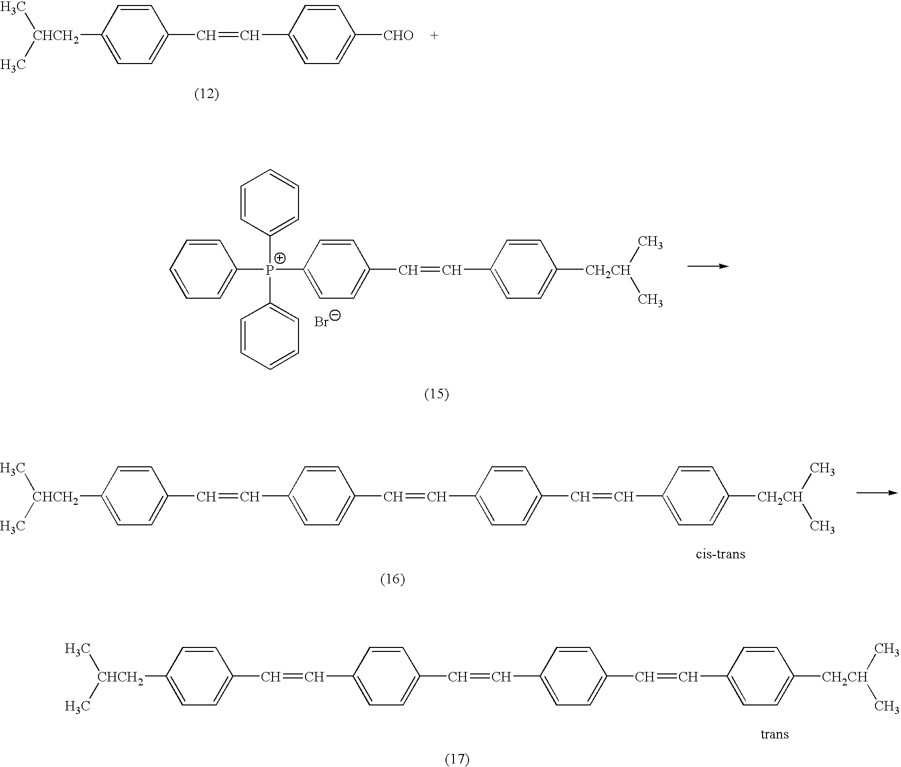

In 90 ml of methanol was dissolved 0.26 g (0.001 mol) of compound (12) to prepare solution A. In 30 ml of methanol was dissolved 0.58 g (0.001 mol) of compound (15) to prepare solution B. Solution B was added to solution A, and 0.19 g of 28% sodium methoxide was slowly added dropwise to the mixture, followed by stirring at 50° C. for 24 hours in a nitrogen atmosphere. After completion of the reaction, the reaction mixture was filtered, and the precipitate collected was washed successively with an ethanol solution and distilled water and dried to give 0.16 g of compound (16) as a yellow solid in a yield of 32.3%.

A mixture of 0.16 g (3×10⁻⁴ mol) of compound (16), 3 mg of iodine, and 13 ml of p-xylene was refluxed at 120° C. for 4 hours, followed by cooling to room temperature. The precipitate thus formed was collected by filtration and washed successively with cold hexane and cold ethanol to yield 0.12 g (75.0%) of compound (17) as a pale yellow solid. The data of ¹H-NMR (CDCl₃) and FT-IR (KBr) analyses for identification are shown in Tables 1 and 2, respectively. The fluorescence spectrum excited at a wavelength of 320 nm showed the maximum peak at 430.8 nm.

TABLE 1
¹H-NMR (CDCl₃)

| δ (ppm) | Splitting | #H | Assignment |
|---|---|---|---|
| 0.8-1.0 | d | 12 | —CH₃ |
| 2.1-2.4 | m | 2 | —CH— |
| 3.6 | d | 4 | —CH₂— |
| 6.9-7.8 | m | 22 | 1 |

TABLE 2
FT-IR (KBr)

| cm⁻¹ | Assignment |
|---|---|
| 843 | C—H out-of-plane bend |
| 1516, 1593 | C═C ring stretch |
| 2866-2951 | aliphatic C—H stretch |
| 3022 | aromatic C—H stretch |

### Example 2

Compounds (21), (22), and (26) were synthesized in accordance with the following reaction scheme.

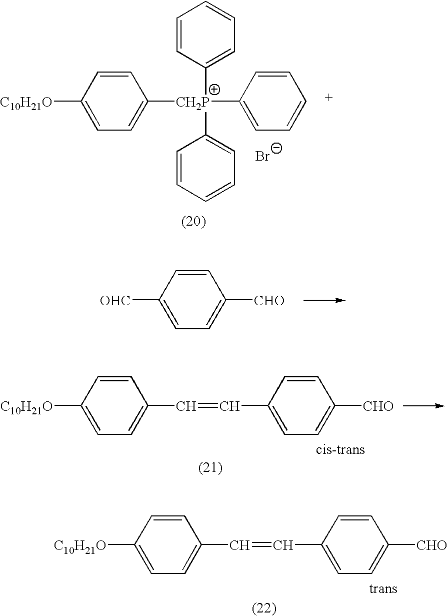

In 100 ml of ethanol were dissolved 4.89 g (0.083 mol) of compound (20) and 16.7 g (0.12 mol) of terephthalaldehyde, and 16g (0.083 mol) of 28% sodium methoxide was added dropwise to the solution, followed by stirring at 50° C. for 24 hours. After completion of the reaction, the reaction mixture was extracted with chloroform. The extract was freed of the solvent by evaporation under reduced pressure and washed with hexane to give compound (21).

A mixture of 8.21 g (0.03 mol) of compound (21), 22.4 mg of iodine, and 40 ml of p-xylene was refluxed at 120° C. for 4 hours. After cooling to room temperature, the precipitate formed was collected by filtration and washed successively with cold hexane and cold ethanol to afford 6.68 g (81.4%) of compound (22) as a pale yellow solid. Compound (26) was then synthesized therefrom as follows. The C₁₀H₂₁O— group in the compound (26) was straight.

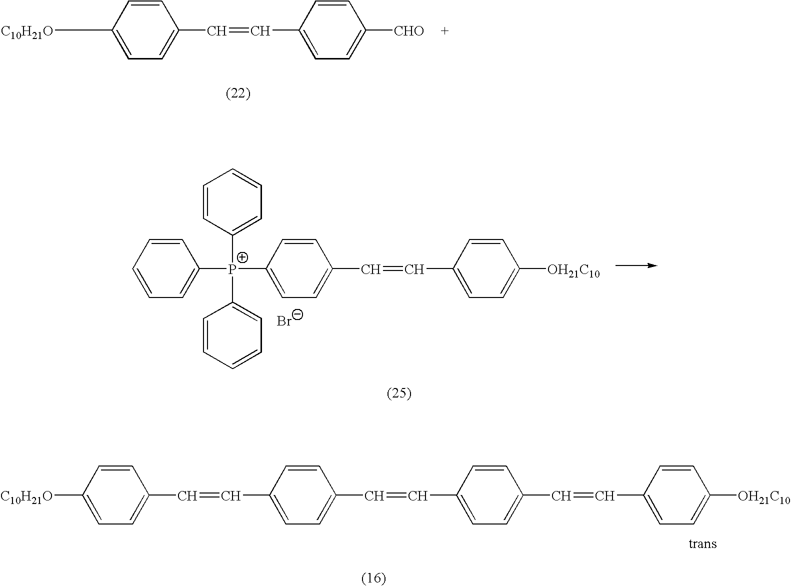

In 20 ml of methanol was dissolved 0.16 g (4.3×10⁻⁴ mol) of compound (22) to prepare solution A. In 50 ml of methanol was dissolved 0.3 g (4.3×10⁻⁴ mol) of compound (25) to prepare solution B. Solution B was added to solution A, and 0.08 g (4.3×10⁻⁴ mol) of 28% sodium methoxide was slowly added thereto dropwise, followed by stirring at 50° C. for 24 hours in a nitrogen atmosphere. After completion of the reaction, the reaction mixture was filtered to collect the precipitate, which was washed with an ethanol solution and dried to give 0.08 g (32.3%) of compound (26) as a yellow solid. The data of ¹H-NMR (CDCl₃) and FT-IR (KBr) analyses for identification are shown in Tables 3 and 4, respectively. The fluorescence spectrum excited at a wavelength of 320 nm showed the maximum peak at 430.10 nm.

TABLE 3
¹H-NMR (CDCl₃)

| δ (ppm) | Splitting | #H | Assignment |
|---|---|---|---|
| 0.8-1.8 | m | 38 | C₈H₁₆, —CH₃ |
| 3.9 | t | 4 | —CH₂—O— |
| 6.9-8.2 | m | 22 | 1 |

TABLE 4
FT-IR (KBr)

| cm⁻¹ | Assignment |
|---|---|
| 833 | C—H out-of-plane bend |
| 1253 | C—O antisymmetric stretch vibration |
| 1604 | C═C ring stretch |
| 2850-2919 | C—H stretch |
| 3772 | aromatic C—H stretch |

### Example 3

Compound (27) was synthesized in accordance with the following reaction scheme. The C₁₀H₂₁O— group in the compound (27) was straight.

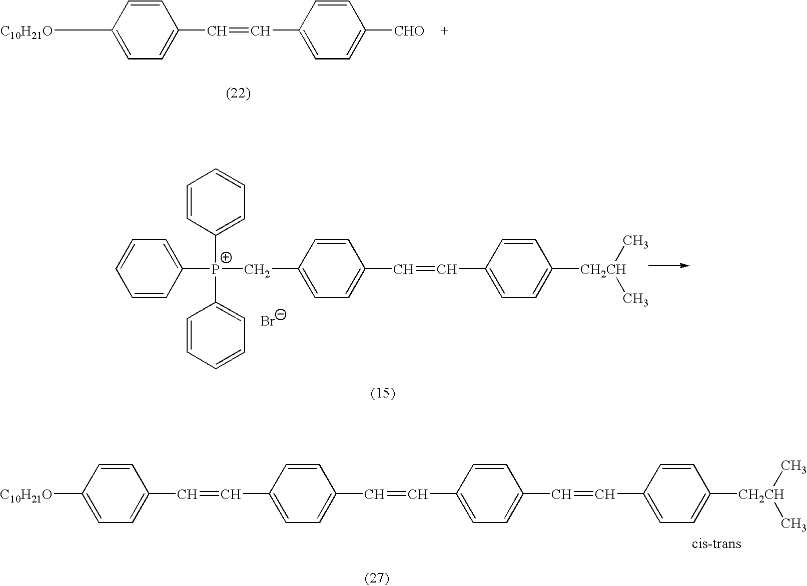

In a mixed solvent of 90 ml of methanol and 10 ml of chloroform was dissolved 0.2 g (5.6×10⁻⁴ mol) of compound (22) to prepare solution A. In 30 ml of methanol was dissolved 0.33 g (5.6×10⁻⁴ mol) of compound (15) to prepare solution B. Solution B was added to solution A, and 0.11 g of 28% sodium methoxide was slowly added thereto dropwise, followed by stirring at 50° C. for 24 hours in a nitrogen atmosphere. After completion of the reaction, the reaction mixture was filtered, and the precipitate collected was washed successively with an ethanol solution and distilled water, and dried to give 0.16 g (47.9%) of compound (27) as a yellow solid. The data of ¹H-NMR (CDCl₃) and FT-IR (KBr) analyses for identification are shown in Tables 5 and 6, respectively. The fluorescence spectrum excited at a wavelength of 320 nm showed the maximum peak at 430.10 nm.

TABLE 5
¹H-NMR (CDCl₃)

| δ (ppm) | Splitting | #H | Assignment |
|---|---|---|---|
| 0.8-0.9 | m | 9 | —CH₃ |
| 1.2-1.3 | m | 16 | —C₈H₁₆— |
| 1.7-1.8 | m | 1 | —CH— |
| 2.4 | d | 2 | —CH₂— |
| 3.9 | t | 2 | —CH₂—O— |
| 6.5-7.8 | m | 22 | 1 |

TABLE 6
FT-IR (KBr)

| cm⁻¹ | Assignment |
|---|---|
| 839 | C—H out-of-plane bend |
| 1252 | C—O antisymmetric stretch vibration |
| 1514, 1597 | C═C ring stretch |
| 2850-2954 | aliphatic C—H stretch |
| 3022 | aromatic C—H stretch |

### Example 4

Compound (29) and compound (30) were synthesized in accordance with the following reaction scheme. The C₇H₁₅— group in these compounds was straight.

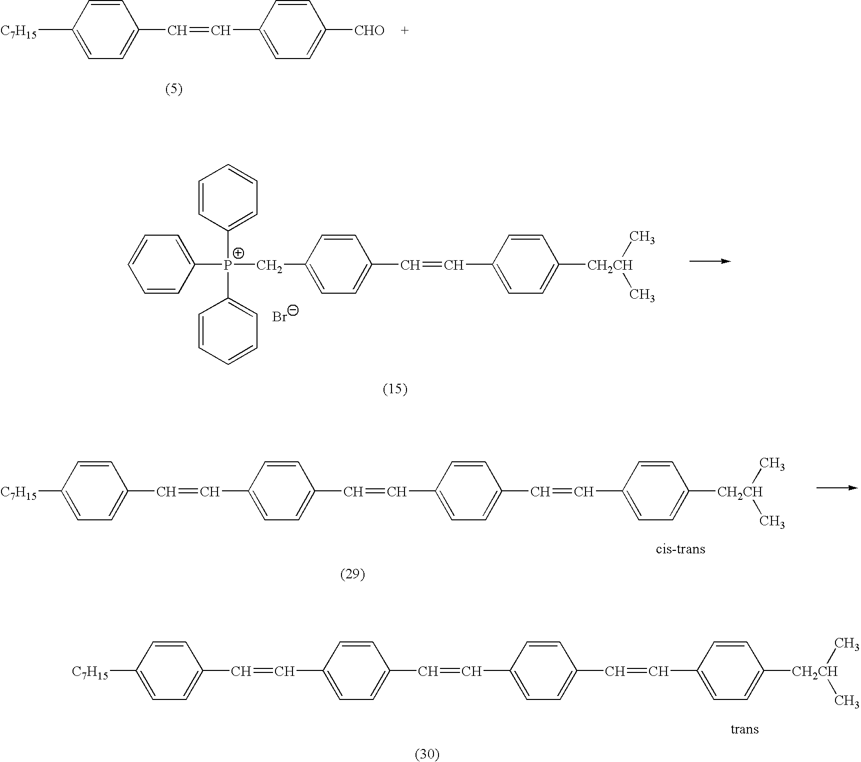

In 30 ml of methanol was dissolved 0.73 g (2.4×10⁻³ mol) of compound (5) to prepare solution A. In 50 ml of methanol was dissolved 1.4 g (2.4×10⁻³ mol) of compound (15) to prepare solution B. Solution B was added to solution A, and 0.46 g of 28% sodium methoxide was slowly added thereto dropwise, followed by stirring at 65° C. for 24 hours in a nitrogen atmosphere. After completion of the reaction, the reaction mixture was filtered, and the precipitate collected was washed successively with an ethanol solution and distilled water and dried to give 0.24 g (11.4%) of compound (29) as a yellow solid.

A mixture of 0.24 g (5.0×10⁻⁴ mol) of compound (29), 1 mg of iodine, and 8 ml of p-xylene was refluxed at 120° C. for 4 hours, followed by cooling to room temperature. The precipitate thus formed was collected by filtration and washed successively with cold hexane and cold ethanol to yield 0.20 g (83.3%) of compound (30) as a yellow solid. The data of ¹H-NMR (CDCl₃) and FT-IR (KBr) analyses for identification are shown in Tables 7 and 8, respectively. The fluorescence spectrum excited at a wavelength of 320 nm showed the maximum peak at 430.10 nm.

TABLE 7
¹H-NMR (CDCl₃)

| δ (ppm) | Splitting | #H | Assignment |
|---|---|---|---|
| 0.8-0.9 | m | 9 | —CH₃ |
| 1.2-1.3 | m | 10 | C₅H₁₀ |
| 1.7-1.8 | m | 1 | —CH— |
| 2.4 | d | 2 | —CH₂— |
| 2.6 | t | 2 | —CH₂— |
| 7.0-7.8 | m | 22 | 1 |

TABLE 8
FT-IR (KBr)

| cm⁻¹ | Assignment |
|---|---|
| 839 | C—H out-of-plane bend |
| 1514, 1597 | C═C ring stretch |
| 2850-2954 | aliphatic C—H stretch |
| 3022 | aromatic C—H stretch |

### Evaluation of Physical Properties as Liquid Crystal Compound

Phase transitions of the compounds obtained in Examples 1 to 3 are shown in Table 9. The compound of Example 4 was confirmed to be a liquid crystal compound having a smectic phase as a homeotropic alignment (perpendicular to the substrate) as a result of observation of transmitted light under a polarizing microscope.

TABLE 9

| | Phase Transition (° C.) |
|---|---|
| Exam- ple 1 | 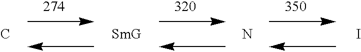 |
| Exam- ple 2 | 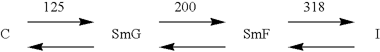 |
| Exam- ple 3 | 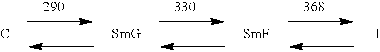 |

Note:
C; crystal, SmG; smectic G phase, SmF; smectic F phase, N; nematic phase, I; isotropic liquid

### Preparation and Evaluation of Liquid Crystal Semiconductor Device

### (1) Organic EL Device

An ITO film (indicated by reference numeral 2 in FIG. 1) was deposited by sputtering to a thickness of 160 nm on a glass substrate (2 mm by 2 mm, 0.7 mm thick; indicated by reference numeral 1 in FIG. 1). PEDOT-PSS was applied thereon by spin coating. The unnecessary part of the applied PEDOT-PSS was removed from the substrate with 2-propanol. The PEDOT-PSS was cured by heating at 150° C. for 30 minutes to form a 0.1 μm thick PEDOT-PSS layer (indicated by reference numeral 3 in FIG. 1).

The substrate was set in a vacuum evaporation system. Thirty milligrams of the styryl derivative synthesized in Example 1 was put in an evaporation boat, and the boat was set in the system at a distance of 15 cm between the substrate and the evaporation material. Vacuum deposition was carried out at room temperature (25° C.) while monitoring the state of evaporation by means of a vacuometer. After completion of the vacuum deposition, nitrogen gas having passed through a desiccant bed was introduced into the vacuum chamber to atmospheric pressure. The substrate having the deposited layer was heat treated at 290° C. for 3 minutes in a substrate heating unit and allowed to cool spontaneously to form a 300 nm thick electroconductive liquid crystal layer (indicated by reference numeral 4 in FIG. 1).

Metallic aluminum was then deposited on the liquid crystal layer by vacuum evaporation to form a 100 nm thick negative electrode layer (indicated by reference numeral 5 in FIG. 1).

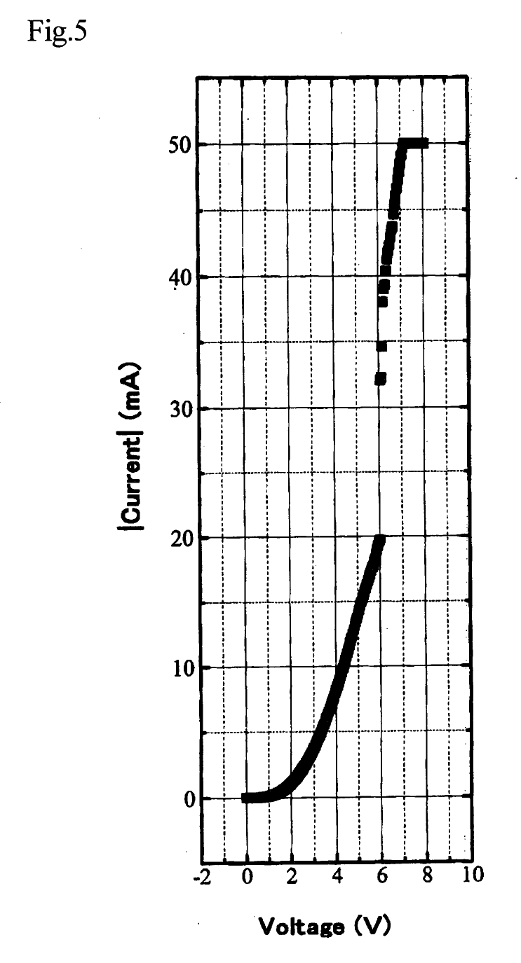

A varying voltage was applied to the resulting device at 25° C., and the amount of the current was measured for every rise in voltage. The results obtained are shown in FIG. 5. It is seen from the results in FIG. 5 that the electroconductive liquid crystal material of the invention exhibits excellent electroconductivity at a low voltage with a threshold voltage of about 5 V in a room temperature (25° C.). When the fluorescence spectrum of the device was observed in the dark, green luminescence was observed.

### (2) TFT

A substrate having a gold drain electrode (indicated by reference numeral 9 in FIG. 3), a gold source electrode (indicated by reference numeral 8 in FIG. 3), and a silicon gate electrode (indicated by reference numeral 7 in FIG. 3) and an evaporation boat containing 30 mg of the styryl derivative synthesized in Example 1 were set obliquely in a vacuum evaporation system at a distance of 15 cm between the substrate and the evaporation material. Oblique vacuum deposition was carried out at room temperature (25° C.) while monitoring the state of evaporation by means of a vacuometer. After completion of the vacuum deposition, nitrogen gas having passed through a desiccant bed was introduced into the vacuum chamber to atmospheric pressure. The substrate having the deposited layer was heat treated at 290° C. for 3 minutes in a substrate heating unit and allowed to cool spontaneously to form a satisfactory electroconductive liquid crystal layer (indicated by reference numeral 11 in FIG. 3).

### INDUSTRIAL APPLICABILITY

As described, the present invention provides a novel liquid crystalline styryl derivative and a process of preparing the same. The styryl derivative of the invention is superior in electrical stability represented by smaller electrical activation energy owing to its longer conjugate structure as compared with an analogous compound having two repeating styryl groups in the formula (1). The styryl derivative is therefore well suited for application to sites near an electrode or the like which undergo a strong electric stimulus.

1. A liquid crystalline styryl derivative represented by general formula (1): 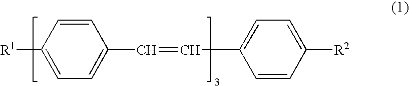
wherein R¹ and R², which may be the same or different,
each represent a straight-chain or branched alkyl group,
a straight-chain or branched alkoxy group, a cyano group,
a nitro group, F, —C(O)O(CH₂)ₘ—CH₃,
—C(O)—(CH₂)ₘ—CH₃, or general formula (2); 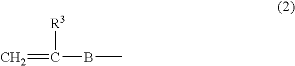
wherein R³ represents a hydrogen atom or a methyl group;
B represents —(CH₂)ₘ—, —(CH₂)ₘ, —, —CO—O—(CH₂)ₘ—,
—CO—O—(CH₂)ₘ—O—, —C₆H₄—CH₂—O— or —CO—; and m represents
an integer of 1 to 18.

2. The liquid crystalline styryl derivative according to claim 1, wherein R¹ and R² in general formula (1), which may be the same or different, each represent a straight-chain or branched alkyl group or a straight-chain or branched alkoxy group.

3. The liquid crystalline styryl derivative according to claim 2, wherein R¹ and R² in general formula (1), which may be the same or different, each represent a branched alkyl or alkoxy group represented by CH₃—(CH₂)ₓ—CH(CH₃)—(CH₂)_{y}—CH₂— or CH₃—(CH₂)ₓ—CH(CH₃)—(CH₂)_{y}—CH₂—O—, respectively, wherein x is an integer of 0 to 7, and y is an integer of 0 to 7.

4. The liquid crystalline styryl derivative according to claim 2, wherein R¹ and R² in general formula (1) each represent an isobutyl group or an isobutoxy group.

5. The liquid crystalline styryl derivative according to claim 2, wherein R¹ and R² in general formula (1) each represent a straight-chain heptyl group or a straight-chain decyloxy group.

6. The liquid crystalline styryl derivative according to claim 1, which is used as an organic semiconductor material.

7. A process of preparing the liquid crystalline styryl derivative according to claim 1, comprising causing a 4-styrylbenzaldehyde compound represented by general formula (3) to react with a phosphonium salt represented by general formula (4): 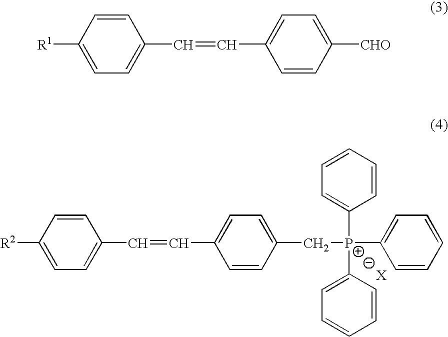
wherein R¹ and R² are as defined in claim 1; and X represents a halogen atom.

8. A liquid crystal semiconductor device comprising a liquid crystal material comprising the liquid crystalline styryl derivative according to claim 1.

9. The liquid crystal semiconductor device according to claim 8, wherein the liquid crystal material is obtained by depositing the liquid crystal material thin film by vacuum evaporation or oblique vacuum evaporation in a room temperature range (from 5° C. to 40° C.) and heating the deposited thin film to a temperature at which the liquid crystal material undergoes phase change to a smectic phase in an inert gas atmosphere.
